(19) United States
(12) Patent Application Publication
(10) Pub. No.: US 2016/0022459 A1
(43) Pub. Date: Jan. 28, 2016

(54) ARTERIAL CONSTRICTOR FOR WEIGHT LOSS TREATMENT

(71) Applicants: Sanja Misra, Rochester, MN (US); MOTARLESS TECHNOLOGIES LLC, Rochester, MN (US)

(72) Inventors: Brian A. Price, Rochester, MN (US); Sanjay Misra, Rochester, MN (US)

(21) Appl. No.: 14/776,470

(22) PCT Filed: Feb. 26, 2014
(86) PCT No.: PCT/US14/18731

§ 371 Date: Sep. 14, 2015

Related U.S. Application Data
(60) Provisional application No. 61/784,915, filed on Mar. 14, 2013.

(51) Int. Cl. A61F 5/00; A61B 5/145; A61B 17/12; A61M 25/09; A61B 19/00; A61B 5/026; A61B 5/00

(52) U.S. Cl. CPC A61F 5/0073; A61B 5/026; A61B 5/14539; A61B 5/4836; A61M 25/09; A61B 19/5244; A61B 17/12009; A61B 2017/00221

(57) ABSTRACT

This document provides methods and devices involved in medical treatment of morbid obesity. For example, this document provides methods and devices for reducing the digestive efficiency of the intestines by decreasing the arterial blood supply to the intestines.

### CROSS-REFERENCE TO RELATED APPLICATION

This application claims priority to U.S. application Ser. No. 61/784,915, filed on Mar. 14, 2013, entitled ARTERIAL CONSTRICTOR FOR WEIGHT LOSS TREATMENT the disclosure of which is incorporated herein by reference.

### BACKGROUND

1. Technical Field

This document relates to methods and devices involved in medical treatment of morbid obesity. For example, this document relates to methods and devices for reducing the digestive efficiency of the intestines by decreasing the arterial blood supply to the intestines.

2. Background Information

While the rate of obesity in the United States is growing fast, the rate of morbid obesity is growing three times faster. In general, obesity means having too much body fat. Morbid obesity refers to individuals who have a body mass index greater than or equal to 35 kg/meter squared.

Morbid obesity is a serious health condition that can interfere with basic physical functions such as breathing or walking. Those who are morbidly obese are at greater risk for illnesses including diabetes, high blood pressure, sleep apnea, gastroesophageal reflux disease, infertility, low back pain, asthma, gallstones, osteoarthritis, heart disease, and cancer. Billions of dollars are spent each year treating millions of individuals around the world suffering from such diseases.

An active lifestyle with plenty of exercise, along with healthy eating, is typically the ideal way to lose weight. Even modest weight loss can measurably improve an obese person's health. However, many people suffering from morbid obesity find it hard to change their eating habits and lifestyle behaviors. Consequently, many morbidly obese individuals find it nearly impossible to lose weight by controlling their diet and exercising.

### SUMMARY

This document provides methods and devices involved in treating of morbid obesity. For example, this document provides methods and devices for reducing the digestive efficiency of the intestines by decreasing the arterial blood supply to the intestines.

In general, one aspect of this document features a method for reducing caloric uptake of a mammal. The method comprises: implanting an arterial constrictor device in the mammal, wherein the arterial constrictor device is configured to adjustably apply a compressive force on an outer surface of an artery that supplies blood to an intestinal organ; and adjusting the compressive force to modulate blood flow to the intestinal organ to a desired level.

In some implementations, the mammal may be a human. The artery that supplies blood to an intestinal organ may be a superior mesenteric artery, celiac axis artery, inferior mesenteric artery, or branches of the arteries. The adjusting the compressive force to modulate the blood flow to the intestinal organ may include periods of time when the compressive force is adjusted to be substantially zero.

In general, another aspect of this document features a system for adjustably constricting an arterial vessel that supplies blood to an intestinal organ in a mammal. The system comprises: a constrictor device configured for implantation in the mammal; and an external controller configured to wirelessly send the control commands for causing the compressive force to be adjusted. The constrictor device is configured to substantially surround an outer periphery of the arterial vessel and to adjustably apply a compressive force on the outer periphery. The constrictor device is configured to wirelessly receive control commands that are capable of causing the compressive force to be increased, and to wirelessly receive control commands that are capable of causing the compressive force to be decreased. The constrictor device is configured to adjust the compressive force in response to the control commands.

In some implementations, the mammal may be a human. The system may further comprise a sensor configured to assess a level of perfusion of the intestinal organ, and to provide to the constrictor device a signal corresponding to the level of perfusion. The sensor may be a pH sensor. The constrictor device may be configured to adjust the compressive force in response to the signal.

In general, another aspect of this document features a system for adjustably constricting an arterial vessel that supplies blood to an intestinal organ in a mammal. The system comprises: a constrictor device configured for implantation in the mammal using a percutaneous catheter-based technique; and an external controller configured to wirelessly send the control commands for causing the compressive force to be adjusted. The constrictor device is configured to at least partially surround an outer periphery of the arterial vessel and to adjustably apply a compressive force on at least a portion of the outer periphery. The constrictor device is configured to wirelessly receive control commands that are capable of causing the compressive force to be increased, and to wirelessly receive control commands that are capable of causing the compressive force to be decreased. The constrictor device is configured to adjust the compressive force in response to the control commands

In various implementations, the mammal may be a human. The system may further comprise a sensor configured to assess a level of perfusion of the intestinal organ and to provide to the constrictor device a signal corresponding to the level of perfusion. The sensor may be a pH sensor. The constrictor device may be configured to adjust the compressive force in response to the signal.

In general, another aspect of this document features a method for percutaneously installing a system for adjustably constricting an arterial vessel that supplies blood to an intestinal organ in a mammal. The method comprises: inserting a guidewire through an opening in the mammal's skin and using an imaging system to maneuver a distal tip of the guidewire to a target location on the arterial vessel;

installing a catheter over the guidewire; inserting at least a portion of the system into the catheter; causing a distal end of the system to emerge from the catheter at the target location, wherein at least a portion of the distal end of the system wraps around at least a portion of a periphery of the arterial vessel; and implanting a control module of the system under the mammal's skin. The constrictor device is configured to partially surround the at least a portion of a periphery of the arterial vessel and to adjustably apply a compressive force on the at least a portion of a periphery. The constrictor device is configured to wirelessly receive control commands that are capable of causing the compressive force to be increased, and to wirelessly receive control commands that are capable of causing the compressive force to be decreased. The constrictor device is configured to adjust the compressive force in response to the control commands.

In various implementations, the mammal may be a human.

In general, another aspect of this document features a system for adjustably constricting an arterial vessel that supplies blood to an intestinal organ in a mammal. The system comprises: a constrictor device configured for implantation in the mammal using a percutaneous catheter-based technique; and an external controller configured to wirelessly send the control commands for causing the electromotive stimulation to be modulated. The constrictor device comprises at least one electrical lead and an energy source. The at least one electrical lead is arranged to be in electrical communication with an outer surface of the arterial vessel. The energy source is arranged to provide an electromotive stimulation through the electrical lead to at least a portion of the outer surface of the arterial vessel. The constrictor device is configured to wirelessly receive control commands that are capable of modulating the electromotive stimulation. The constrictor device is configured to adjust the electromotive stimulation in response to the control commands

In various implementations, the mammal may be a human.

In general, another aspect of this document features method for reducing caloric uptake of a mammal. The method comprises: implanting an arterial constrictor device in the mammal, wherein the arterial constrictor device is configured to adjustably apply an electromotive stimulation on an outer surface of an artery that supplies blood to an intestinal organ; and adjusting the electromotive stimulation to modulate blood flow to the intestinal organ to a desired level.

In various implementations, the mammal may be a human. The artery that supplies blood to an intestinal organ may be a superior mesenteric artery, celiac axis artery, inferior mesenteric artery, or branches of the arteries.

Particular embodiments of the subject matter described in this document can be implemented to realize one or more of the following advantages. In some embodiments, the methods and systems provided herein can cause weight loss by reducing the caloric absorption of an individual. In some embodiments, the systems provided herein can be conveniently adjustable to enable modulation of the arterial blood flow to an intestinal organ to control the digestive efficiency, while balancing the aggressiveness of the treatment with monitoring the health and comfort of the individual. In some embodiments, additional features are provided, such as a user-operated controller device for decreasing the constriction of the arterial blood flow, and a perfusion sensor for monitoring the level of perfusion of the intestinal organ that experiences the reduced arterial blood supply.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an anatomical diagram of a portion of a human intestinal region including the arterial vessel structure.

FIG. 2 is the anatomical diagram of FIG. 1 with schematic representations of arterial band-constrictor devices applied at various exemplary locations on the arterial vessels.

FIG. 3 is a schematic representation of an exemplary embodiment of an implanted arterial band-constrictor device in wireless communications with an external controller.

FIG. 4 is a schematic representation of an exemplary embodiment of an arterial band-constrictor device.

FIG. 5 is a schematic representation of another exemplary embodiment of an arterial band-constrictor device.

FIG. 6 is a schematic representation of another exemplary embodiment of an arterial constrictor device.

FIG. 7 is a flowchart of an exemplary method for treating morbid obesity.

FIG. 8A is an anatomical diagram depicting the installation of an arterial constrictor device that can be placed surgically or using image guided assistance.

FIG. 8B is a schematic representation of another exemplary embodiment of an arterial constrictor device that can be placed surgically or using image guided assistance.

FIG. 8C is a schematic representation of another exemplary embodiment of an arterial constrictor device that can be placed surgically or using image guided assistance.

FIG. 9 is a flowchart of an exemplary method for installing an arterial constrictor device using minimally invasive techniques.

Like reference numbers represent corresponding parts throughout.

### DETAILED DESCRIPTION

This document provides methods and devices involved in treating of morbid obesity. For example, this document provides methods and devices for reducing the digestive efficiency of the intestines by decreasing the arterial blood supply to the small intestines.

As described herein, reducing the arterial blood supply to the intestines can reduce the efficiency of the digestive process, and can result in reduced caloric absorption or uptake. In some cases, a reduction of caloric uptake may be associated with a resulting reduction in appetite or caloric intake. In some cases, the methods and devices provided herein can be used to induce weight loss and/or treat obesity. For example, an artery constrictor system provided herein can be used to reduce the arterial blood supply to the small intestines in a manner that reduces the efficiency of the digestive process, and causes a reduction in the absorption of compounds from the food that an individual has consumed.

The methods and systems provided herein can be used to constrict the arterial blood supply of any appropriate mammal and/or can be used to reduce the body weight of any appropriate mammal. For example, the methods and systems provided herein can be used to constrict the arterial blood supply to a human intestine or to reduce the body weight of a human (e.g., a human suffering from morbid obesity).

Any appropriate intestinal artery can be constricted to reduce the arterial blood flow to the intestines, and/or can be used to reduce the body weight of a mammal. For example, the arterial blood flow through intestinal arteries such as the superior mesenteric artery, or the celiac axis, or a branch of the arteries can be constricted to reduce the arterial blood flow to the intestines, and/or can be used to reduce the body weight of a mammal. In some cases, the arterial blood flow through the inferior pancreaticoduodenal artery, the jejunal arteries, the ileal arteries, or a combination thereof can be constricted to reduce the arterial blood flow to the intestines, and/or can be used to reduce the body weight of a mammal.

Any appropriate medical device system for constricting the arterial blood flow to the intestines can be used in accordance with the methods provided herein to reduce the body weight of a mammal. For example, one or more arterial constrictor device systems configured to reduce the arterial blood flow to the intestines can be used to reduce the body weight of a mammal by implanting such arterial constrictor device systems to surround the periphery of one or more intestinal arteries. In some cases, one or more arterial constrictor device systems can be configured to restrict the arterial blood flow through the superior mesenteric artery that supplies portions of the small intestine with blood. In some cases, an arterial constrictor device system can be located externally and on the periphery of an artery to provide a radially directed constricting force on the outer wall of the artery. For example, an arterial constrictor device can surround the superior mesenteric artery and provide a radially directed force to partially restrict and/or occlude the inner diameter of the artery to reduce blood flow therethrough.

Any appropriate construction of a constrictor system can be used to reduce the diameter of an intestinal artery to reduce blood flow therethrough. For example, one embodiment can include a peripheral band with an inflatable inner annulus to apply a radial force on the periphery of an artery. In another example embodiment, an inflatable annulus is generally a semi-circle. That is, the inflatable annulus surrounds a portion of the periphery of an artery. In some cases, a saline inflation fluid can be provided to inflate the inner annulus. In some cases, an implanted pump system can pressurize the inflation fluid to inflate and/or deflate the inflatable annulus. The inflation of the inner annulus by the pump can provide a radially directed force to restrict or occlude partially the inner diameter of an artery to restrict blood flow and/or induce weight loss.

In some cases, an arterial band-constrictor system can include a jacketed motorized mechanical clamping mechanism installed around the periphery of an intestinal artery. In some cases, a reversible DC motor can provide the motive force for actuating a mechanical band clamp to increase and/or decrease the diameter of the clamp device. For example, a jacketed motorized mechanical band clamp can be actuated to apply a radial force to the outer periphery of an artery to constrict the artery and to reduce blood flow therethrough to induce weight loss.

In some cases, an arterial constrictor clamp system can be used to reduce the diameter of an intestinal artery to reduce blood flow therethrough. For example, one embodiment can include a clamp with one or more inflatable pads to apply a compressive force on the surface of an artery. In some cases, a saline inflation fluid can be provided to inflate the inner pads. In some cases, an implanted pump system can pressurize the inflation fluid to inflate and/or deflate the inflatable pads. The inflation of the inner pads by the pump can provide a compressive directed force to restrict or occlude partially the inner diameter of an artery to restrict blood flow and/or induce weight loss.

In some cases, an arterial constrictor actuator is an electrical lead that is wrapped onto the periphery of an intestinal artery. The electrical lead can be supplied with electrical current and controlled by an implantable electrical actuator that includes a battery pack. When electrical pulses are transferred from the electrical lead to the artery, a restriction of blood flow can result from contraction of the artery.

The systems provided herein for constricting arterial blood flow through intestinal arteries can be installed using a variety of techniques. In some cases, an open-surgery technique can be used to install the systems. In some cases, the systems can be installed percutaneously using catheter-based minimally invasive techniques with image guidance.

In some cases, the methods and systems provided herein can adjustably constrict the arterial blood supply of any appropriate mammal and/or can be used to reduce the body weight of any appropriate mammal. In some embodiments, the amount of compressive force directed on the outer surface of an artery can be adjusted by a physician in accordance with a treatment plan to increase and/or to decrease the arterial blood flow to the intestines. Such adjustment can provide a therapy that is effective for inducing weight loss, while not constricting blood flow to an extent that is potentially detrimental to the patient. In some cases, the methods and systems provided herein can provide a convenient process for adjusting the amount of constriction on an artery. For example, a wireless interface between an external controller and an implanted arterial constrictor system can be used to provide a convenient method for adjusting or modulating the amount of constriction on an intestinal artery.

In some cases, the methods and systems provided herein can reversibly constrict the arterial blood supply of a mammal. For example, a device provided herein for constricting an arterial vessel by applying a compressive force to the outer surface of an artery can also be reversed to alleviate the application of such a compressive force. In some cases, an interface, such as a wireless interface, can be used to partially or fully remove the application of a compressive force to the outer surface of an artery supplying blood to the intestines. In such a manner, the methods and systems provided herein are reversible, and can be administered and/or de-administered in keeping with a patient's and physician's desires.

In some cases, additional patient safety features can be included with the methods and systems provided herein. For example, some embodiments can include a sensor for sensing intestinal perfusion and/or ischemia, e.g., a pH sensor to sense ischemia. Such sensor devices can provide feedback to a controller of the arterial constrictor system to reduce (as needed) the amount of compressive force applied to an artery by the system, and/or to relieve ischemia as needed.

In some cases, the patient can be provided with a transmitter that can be used by the patient to reduce the compressive from the arterial constrictor, but not to increase it. Such a transmitter can be a useful safety device in the event that the patient experiences discomfort or any other adverse effects associated with a constricted intestinal blood supply. In the event of adverse effects, the patient can use the transmitter to reduce the constriction to alleviate the discomfort. As an additional safety feature, the patient transmitter can be prevented from increasing the amount of constriction from the arterial constrictor system, so that the patient cannot improperly or inadvertently apply an excess compressive force to an intestinal artery.

In some cases, the arterial constrictor is configured with a maximum constriction limit to prevent the arterial constrictor from exerting excess constriction on an artery. For example, in some cases the arterial constrictor can be prevented from fully occluding the artery on which it is located. In some cases, the arterial constrictor can be prevented from occluding the artery on which it is located at a level that is a harmful level of occlusion. In some cases, the arterial constrictor can include mechanical configurations to establish a maximum constriction limit. Such features can prevent excess constriction in the event of an inadvertent adjustment of the constriction level, and/or in the event of a device malfunction.

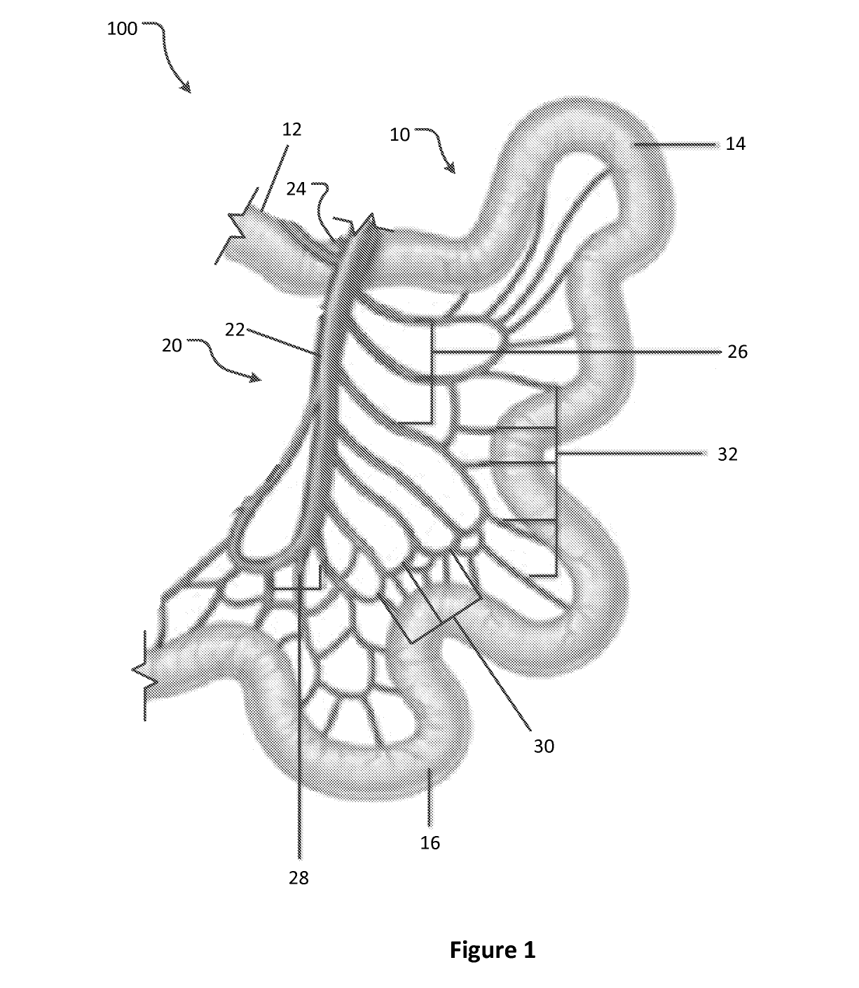

With reference to FIG. 1, the anatomy of the human small bowel 100 includes a small intestine 10 and an arterial network 20 that supplies blood to small intestine 10. Small intestine 10 is a long, narrow, coiled tube extending between the stomach and the large intestine. Small intestine 10 can be approximately twenty feet long in an adult human. Small intestine 10 is where most digestion and absorption of food takes place. In small intestine 10, food is chemically decomposed via reactions with water and with other secretions from the liver, pancreas, and other intestinal glands. In small intestine 10, tiny projections called villi absorb the end products of digestion.

The anatomy of small intestine 10 includes three intestinal portions. A first portion is the duodenum 12. Duodenum 12 is attached to the outlet of the stomach and is approximately 10-15 inches long in an adult human. Duodenum 12 is largely responsible for the breakdown of food in small intestine 10. Duodenum 12 also regulates the rate of emptying of food from the stomach.

The middle portion of small intestine 10 is the jejunum 14. Jejunum 14 is the longest portion of small intestine 10, comprising approximately one-half of the entire length of small intestine 10. The majority of absorption of nutrients takes place in jejunum 14. The mucous membrane on the inner surface of jejunum 14 is covered with hair-like projections called villi. Villi are involved in the absorption or uptake of nutrients such as proteins, carbohydrates, amino acid, sugar, fatty acid particles, vitamins, minerals, electrolytes, and water. Villi contain blood capillaries that are part of the microcirculation system of a human. Reduction of blood supply to the villi can result in a reduction in the amount of compounds adsorbed from food consumed by an individual. Villi are largest and most numerous in the duodenum 12 and jejunum 14, and become fewer and smaller in the ileum 16.

The final portion of small intestine 10 is the ileum 16, which connects to and terminates at the large intestine. Ileum 10 mainly absorbs vitamin B12 and bile salts. Any food that remains undigested and unabsorbed by ileum 16 passes into the large intestine.

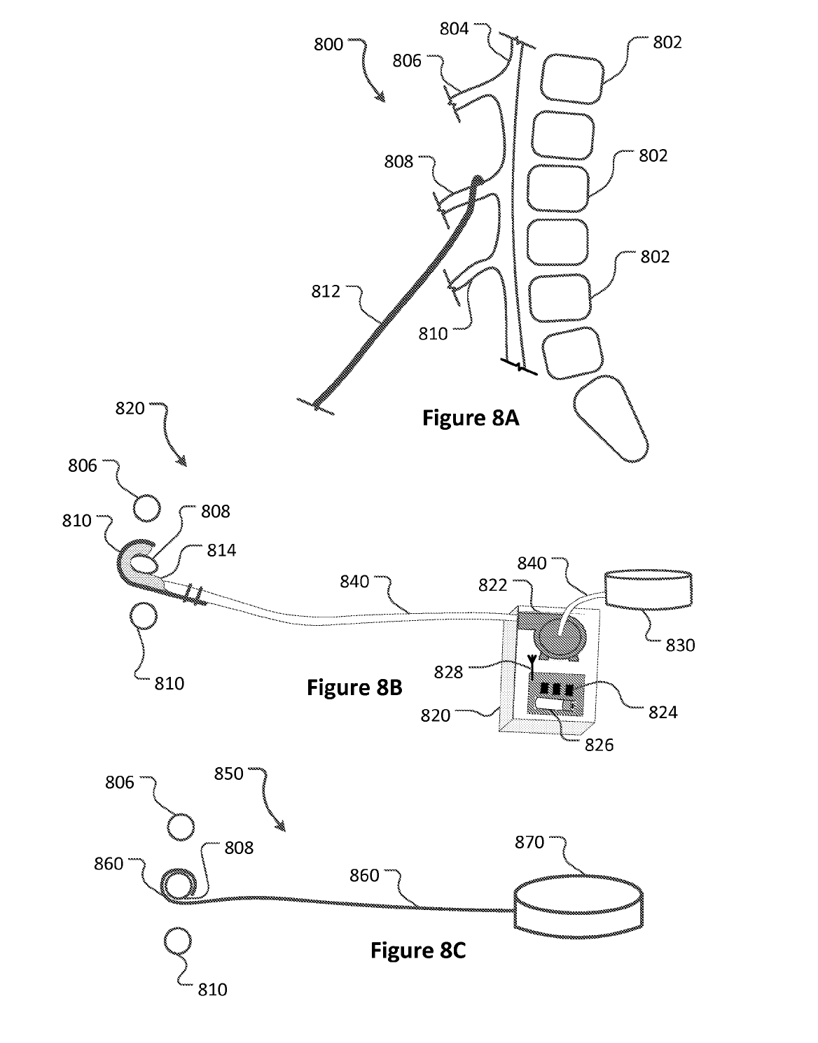

Arterial network 20 supplies oxygen-rich blood to small intestine 10 to support digestion of food and the absorption of nutrients from the food. Arterial network 20 includes the superior mesenteric artery (“SMA”) 22 and its branches. SMA 22 receives oxygenated blood from the abdominal aorta (refer to FIG. 8A). SMA 22 divides its blood flow into various branches that supply the large and small intestines 10.

Duodenum 12 receives arterial blood from two different sources. The upper portion of Duodenum 12 attached to the stomach receives arterial blood from the gastroduodenal artery and its branch the superior pancreaticoduodenal artery. The lower portion of duodenum 12 receives its arterial supply from the inferior pancreaticoduodenal artery 24, which is a branch of SMA 22.

Jejunum 14 and ileum 16 are supplied arterial blood from branches off the left side (from the perspective of the patient) of SMA 22. Approximately 15-18 branches originate from SMA 22 to supply jejunum 14 and ileum 16. These branches unite to form loops or arches called arterial arcades 30. From arterial arcades 30, vasa recta 32 originate that connect to vessels to the walls of jejunum 14 and ileum 16. Jejunum 14 receives the majority of its arterial blood from jejunal arteries 26. Ileum 16 receives the majority of its arterial blood from ileal arteries 30.

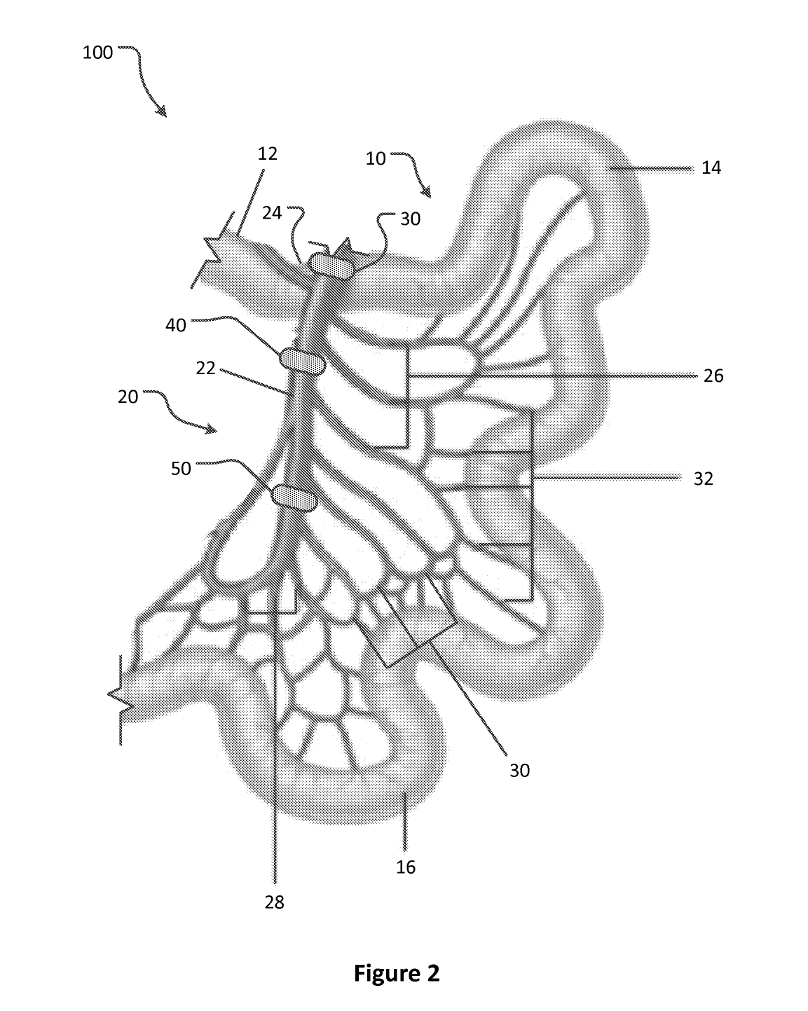

With reference to FIG. 2, a human small bowel 100 is shown with schematically represented arterial band-constrictors located at locations 30, 40, and 50 on SMA 22. In addition to locations on SMA 22, the use of arterial band-constrictors at locations on the gastroduodenal artery, and its branch the superior pancreaticoduodenal artery, are also envisioned. In some implementations, a single arterial band-constrictor device is installed on an intestinal artery to reduce blood flow to the intestines. In some implementations, two or more arterial band-constrictor devices are installed at various positions on the arterial network 20 to reduce blood flow to the intestines.

For example, a single arterial band-constrictor device can be installed at example location 30. In such a case, a band-constrictor device at location 30 can constrict an upper portion of SMA 22 to reduce the arterial blood flow to substantially all portions of jejunum 14 and ileum 16, while leaving the duodenum 12 unaffected.

In some cases, a single arterial band-constrictor device can be installed at location 40. At location 40, a band-constrictor device can reduce arterial blood flow to lower portions of jejunum 14 and substantially all of ileum 16, while leaving blood flow to duodenum 12 and upper jejunum 14 unaffected. Placement of a band-constrictor device at location 40 may represent a less aggressive treatment modality as compared to location 30.

In some cases, a single arterial band-constrictor device can be installed at location 50. At location 50, a band-constrictor device can reduce arterial blood flow to ileum 16 while leaving blood flow to duodenum 12 and jejunum 14 unaffected. Placement of a band-constrictor device at location 50 may represent a less aggressive treatment modality as compared to locations 30 and/or 40.

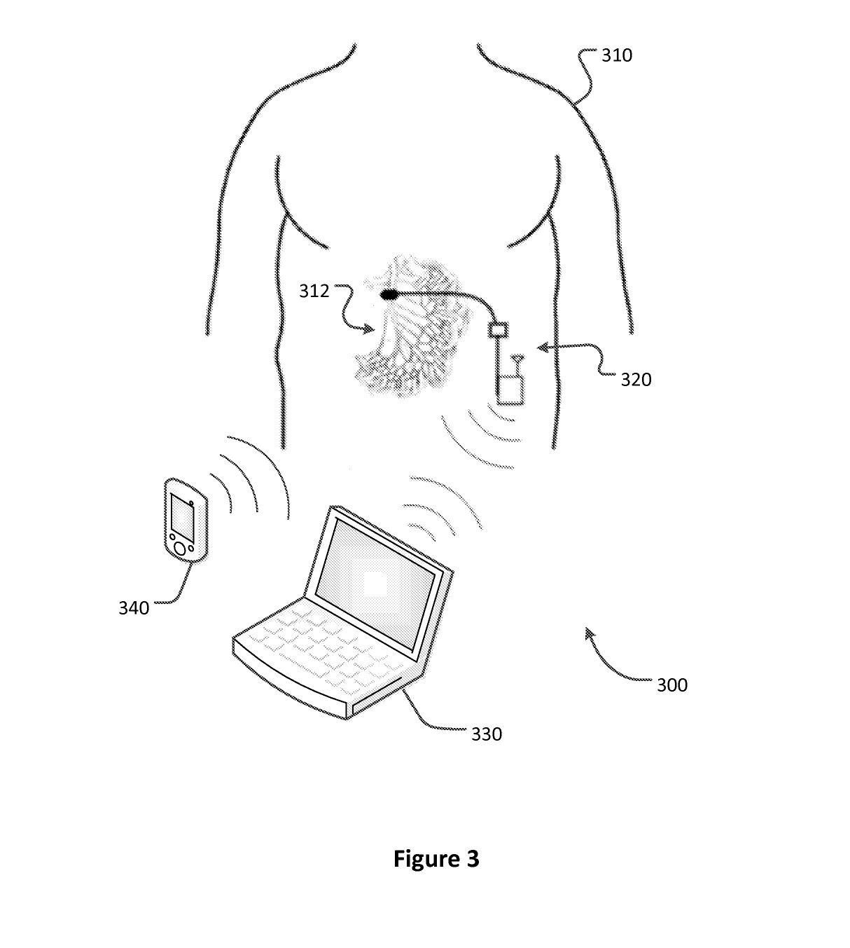

In some cases, two or more arterial band-constrictor devices are installed, for example, at locations 30, 40, and/or 50. When two or more arterial band-constrictor devices are installed, a contoured arterial blood supply pattern can be created in accordance with a particular treatment plan desired by a physician for a particular patient. For example, an arterial band-constrictor device installed at location 30 can reduce the arterial blood flow through the majority of arterial network 20 that supplies small intestine 10. In addition, an arterial band-constrictor device can be installed at location 40 or 50 to further reduce the arterial blood flow to certain portions of small intestine 10, e.g., lower jejunum 14 and/or ileum 16. A multitude of combinations of arterial band-constrictor device locations, and amounts of constriction at the locations, are possible to create a desired profile of arterial blood flow to the intestinal organs. With reference to FIG. 3, an arterial band-constrictor system 300 includes an arterial band-constrictor device 320, implanted within a patient 310, a wireless external controller 330, and an optional wireless patient controller 340. In general, wireless external controller 330, which is configured for operation by a physician, can wirelessly communicate with arterial band-constrictor device 320 that is totally implanted in patient 310. In this fashion, a physician can use wireless external controller 330 to send control commands to arterial band-constrictor device 320 that cause arterial band-constrictor device 320 to increase or decrease the radial forces applied to the periphery of the artery that it surrounds, and to thereby increase or decrease arterial blood flow to the small bowel 312 of patient 310.

Wireless external controller 330 and arterial band-constrictor device 320 can communicate using a variety of wireless technologies. For example, in some cases, radio frequency (“RF”) communications can be used. In some cases, Bluetooth, infrared, ultrasound, and other various suitable wireless modes of device communication can be used for wireless communications between wireless external controller 330 and arterial band-constrictor device 320. Optional wireless patient controller 340 can also use such types of wireless communication technologies.

Wireless external controller 330 provides a convenient way for controlling the amount of constriction to the arterial blood flow of small bowel 312 of patient 310. In some cases, after initial implantation of arterial band-constrictor device 320 a physician may desire to gradually increase the constriction on the arterial blood flow of small bowel 312. For example, after implantation, the physician may wish to initially not apply any radial force to the one or more arteries to which arterial band-constrictor device 320 is applied. Such a treatment plan may be suitable in view of swelling of the internal organs of patient 310 resulting from the implantation surgery.

After a period of time, such as a few days for example, the physician may desire to apply an initial small amount of constriction from arterial band-constrictor device 320 to the artery, which it surrounds. To do so, the physician can wirelessly send such a command from wireless external controller 330 to arterial band-constrictor device 320. In such a fashion, no invasiveness to the body of patient 310 is required. After applying an initial small amount of constriction to the arterial supply of small bowel 312, the physician can monitor the status of patient 310. For example, patient 310 may experience weight loss because of reduced absorption of nutrients from consumed food. In some cases, patient 310 may experience no noticeable changes to their digestion, and experience no weight loss at this stage.

In some cases, if patient 310 has not begun to experience weight loss, a physician may desire to increase the constriction of arterial band-constrictor device 320 on the arterial blood supply for small bowel 312. In that case, the physician can conveniently increase the radial force applied by arterial band-constrictor device 320 by sending a command from wireless external controller 330. No invasiveness to patient 310 is required to make such an adjustment. In this fashion, a physician can control arterial band-constrictor device 320 with minimal inconvenience to patient 310. After making such an adjustment to arterial band-constrictor device 320, the physician can once again monitor the status of patient 310 to determine whether the amount of radial force being applied by arterial band-constrictor device 320 to the periphery of the artery is a desired amount. In some cases, patient 310 may exhibit weight loss. In some cases, patient 310 may not experience any changes and further adjustments to arterial band-constrictor device 320 may be desirable. In some cases, however, patient 310 may experience abdominal discomfort or digestive issues. In such cases, it may be desirable to reduce the amount of restriction by arterial band-constrictor device 320 on the arterial supply to small bowel 312. Such adjustments can be made using wireless external controller 330, and optionally using wireless patient controller 340.

Arterial band-constrictor system 300 can optionally be controlled by wireless patient controller 340. For example, in the event of discomfort to patient 310, patient 310 may be able to relieve some or all of the constriction applied by arterial band-constrictor device 320 using wireless patient controller 340. In some cases, wireless patient controller 340 can thereby provide a safety feature to relieve discomfort and prevent potential adverse health effects resulting from an artery being overly constricted by arterial band-constrictor device 320. In some cases, wireless patient controller 340 can only de-constrict the arterial blood flow by reducing the radial force applied to the artery by arterial band-constrictor device 320, and it cannot increase the constriction to the artery. Such a feature can ensure that patient 310 cannot improperly or inadvertently apply an excess radial force to an intestinal artery.

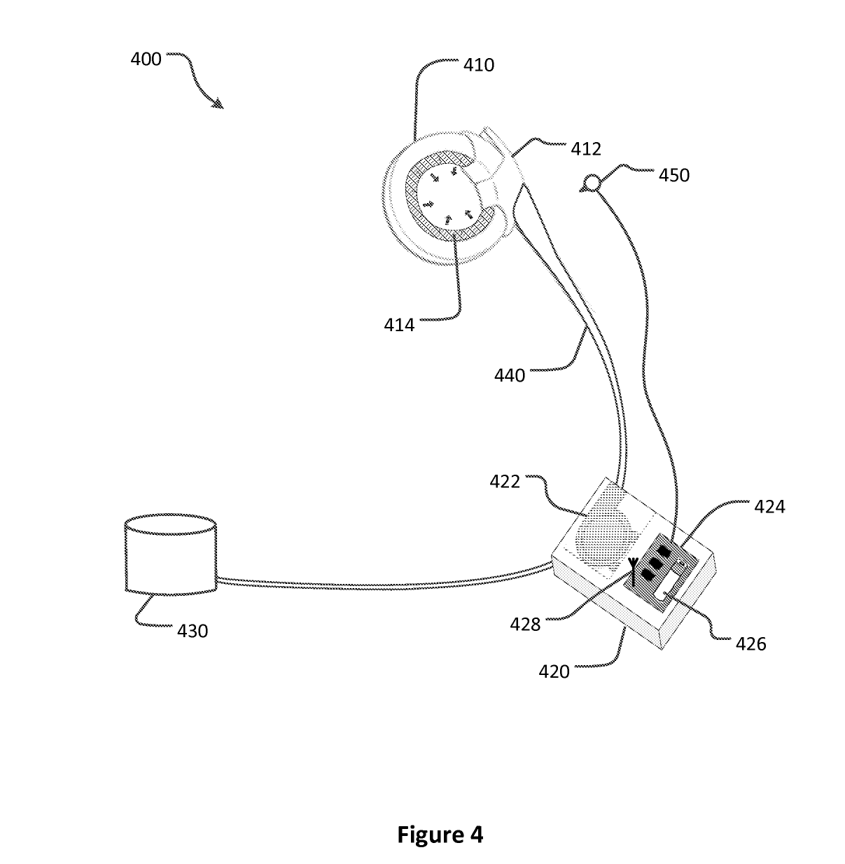

With reference to FIG. 4, an arterial band-constrictor system 400 includes a band 410, a controller 420, and a reservoir 430, that are interconnected by a flexible tubing 440. In some cases, arterial band-constrictor system 400 is fully implantable in a patient's body. As such, all materials are biocompatible.

Band 410 can be configured to surround an artery, and to apply a radial force on the periphery of the artery. The radial force applied on the periphery of the artery can cause a constriction of the blood flow through the artery. In the case of an intestinal artery, the constriction of blood flow through the artery can result in a reduction of digestive efficiency and food adsorption, and consequently cause weight loss.

In some cases, band 410 includes a clasp 412 and an inflatable annulus 414. Clasp 412 is configured to allow band 410 to have an open portion for installation of band 410 around an artery when clasp 412 is open. Clasp 412 allows band 410 to be installed on an artery without the need for severing the artery. After installing band 410 on an artery, clasp 412 is closed to secure band 410 on the artery.

Inflatable annulus 414 is a surface in the inner periphery of band 410 that contacts at least a portion of the outer periphery of the artery on which band 410 is installed. Inflatable annulus 414 is a flexible, balloon-like material, and can comprise silicon and other suitable materials. Inflatable annulus 414 can be inflated by pumping fluid to the interior of inflatable annulus 414. Inflatable annulus 414 can also be deflated by pumping fluid out of the interior of inflatable annulus 414 or by merely relieving the fluid pressure from inside of inflatable annulus 414. By inflating inflatable annulus 414, radial forces can be applied to the outer periphery of an artery that band 410 surrounds. As such, band 410 is sized according to the size of the particular artery on which band 410 will be installed.

The fluid used to inflate inflatable annulus 414 can be provided from a reservoir 430. Reservoir 430 can be implanted beneath the surface of the skin within the patient. Reservoir 430 can be flexible so that as fluid flows from reservoir 430 to inflatable annulus 414, reservoir 430 can collapse in response to having a lower volume of fluid. Reservoir 430 can also flexibly receive fluid from inflatable annulus 414 when inflatable annulus 414 is deflated. In some cases, the fluid used for inflation can be a saline solution, or any suitable biocompatible fluid.

The inflation and deflation of inflatable annulus 414 can be accomplished by the actions of a controller 420. Controller 420 can include a reversible pump 422, a microprocessor 424, a power source 426, and an antenna 428.

Reversible pump 422 can pressurize the inflation fluid to create pressure differentials between reservoir 430 and inflatable annulus 414. Reversible pump 422 can operate to cause inflation fluid to flow from reservoir 430 to inflatable annulus 414, and to flow from inflatable annulus 414 to reservoir 430. Reversible pump 422 can also maintain a constant pressure differential between reservoir 430 and inflatable annulus 414. For example, when inflatable annulus 414 is pressurized in comparison to reservoir 430, reversible pump 422 can maintain the pressure differential with no substantial pressure decay over time.

Microprocessor 424 can control the operations of reversible pump 422. Microprocessor 424 can receive control commands from external controllers via antenna 428. Microprocessor 424 can provide a power switching function by directing electrical power from power source 426 (e.g., lithium-iodine batteries, lithium-ion batteries, and the like) to reversible pump 422 to actuate reversible pump 422 in the desired flow direction. In some cases, the power source 426 can be one or more rechargeable batteries and the rechargeable batteries can be recharged using a wireless induction charging system.

Optionally, a safety electrode 450 can be in electrical communication with microprocessor 424. In some cases, safety electrode 450 can provide feedback to microprocessor 424 regarding the status of arterial perfusion of intestines affected by arterial band-constrictor device 400. For example, safety electrode 450 can be a pH sensor that measures the pH of the intestinal tissue. When pH of tissue drops below a threshold level, it can be an indicator that the blood flow to the tissue is insufficient to prevent ischemia, and that the amount of constriction on the artery should be reduced. In some cases, safety electrode 450 can be another suitable type of perfusion-monitoring sensor that can provide feedback to microprocessor 424 regarding the extent of perfusion of the intestinal tissues affected by arterial band-constrictor device 400. With the feedback provided by the optional safety electrode 450, microprocessor 424 can respond by taking various countermeasures as appropriate. In some cases, microprocessor 424 can actuate reversible pump 422 to decrease the radial force applied by inflatable annulus 414 on the artery it surrounds. In some cases, microprocessor 424 can trigger an alarm that can be received by wireless controllers, such as those described in reference to FIG. 3. In some cases, both such actions, and others, can be initiated by microprocessor 424.

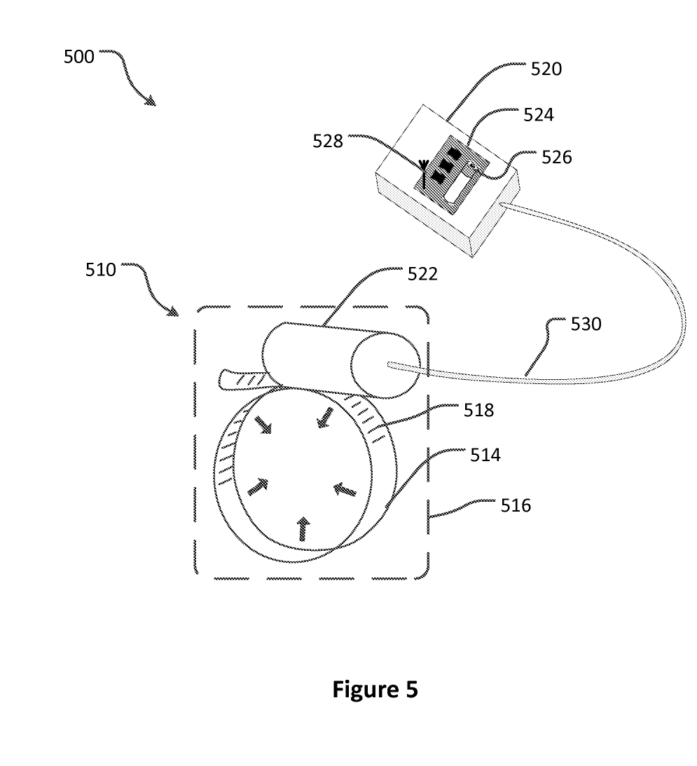

With reference to FIG. 5, another arterial band-constrictor system 500 can include a band-constrictor assembly 510 and a controller 520, that are interconnected by an electrical cable 530. In general, band-constrictor assembly 510 and controller 520 are implanted in a patient in a configuration to constrict arterial blood flow through an artery that supplies an intestinal organ.

Band-constrictor assembly 510 can include a band 514 and a reversible DC motor 522 contained within a jacket 516 (represented schematically). Jacket 516 can enclose the surfaces of band 514 and reversible DC motor 522 to configure the band-constrictor assembly 510 for implantation in a patient. In some cases, band 514 comprises a biocompatible metallic material such as stainless steel, titanium, nitinol, and the like. In some cases, band 514 comprises a flexible polymeric material. Band 514 can include slots 518.

Reversible DC motor 522 can be fixedly coupled to an end of band 514, while being movably coupled to another portion of band 514. In some cases, reversible DC motor 522 can be movably coupled to band 514 via a worm gear with teeth that engage with slots 518. In such a configuration, actuation of reversible DC motor 522 can cause the diameter of band 514 to increase or decrease. When band 514 surrounds an artery, the increase or decrease of the diameter of band 514 can cause a constriction or de-constriction of the artery.

Reversible DC motor 522 can be in electrical communication with controller 520 via electrical cable 530. Controller 520 can include microprocessor 524, power source 526, and antenna 528. Microprocessor 524 can control the operations of reversible DC motor 522. Microprocessor 524 can receive control commands from external controllers via antenna 528. Microprocessor 524 can provide a power switching function by directing electrical power from power source 526 (e.g., lithium-iodine batteries, lithium-ion batteries, and the like) to reversible DC motor 522 to actuate band constrictor assembly 510 in the desired direction to constrict or de-constrict the artery that band 514 surrounds. As with the embodiment described in reference to FIG. 4, additional safety sensors may be included with example arterial band-constrictor device 500.

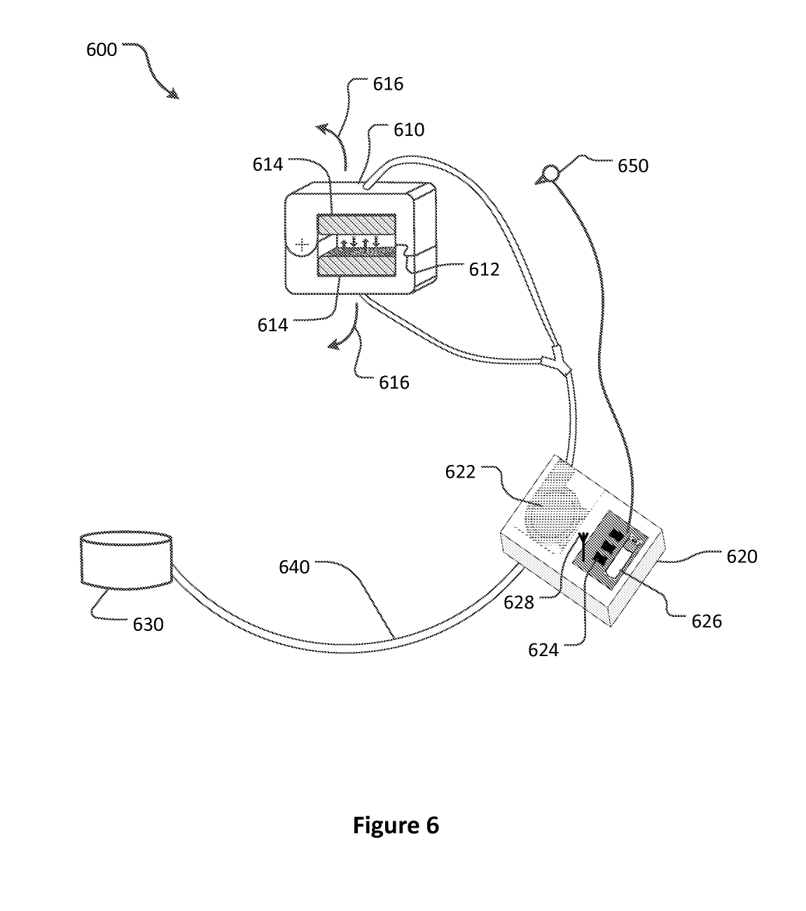

With reference to FIG. 6, an arterial constrictor system 600 includes a clamp 610, a controller 620, and a reservoir 630, that are interconnected by a network of flexible tubing 640. In some cases, arterial constrictor system 600 is fully implantable in a patient's body. As such, all materials are biocompatible.

Clamp 610 can be configured to surround an artery, and to apply a compressive force on the surfaces of the artery. The compressive force applied on the surfaces of the artery can cause a constriction of the blood flow through the artery. In the case of an intestinal artery, the constriction of blood flow through the artery can result in a reduction of digestive efficiency and food adsorption, and consequently cause weight loss.

In some cases, clamp 610 includes a clasp 612 and one or more inflatable pads 614. Clasp 612 can be configured to allow clamp 610 to have an open configuration for installation of clamp 610 around an artery when clasp 612 is open. That is, in some cases, clamp 610 can be pivoted open as indicated by arrows 616, and placed around a target artery to be treated. Clasp 612 allows clamp 610 to be installed on an artery without the need for severing the artery. After installing clamp 610 on an artery, clasp 612 can be closed to secure clamp 610 on the artery.

The one or more inflatable pads 614 are the surface(s) in the inner region of clamp 610 that contact at least a portion of the outer surface of the artery on which clamp 610 is installed. In some cases, a single inflatable pad 614 is included in clamp 610. In some cases, two (2) inflatable pads 614 are included in clamp 610. Inflatable pads 614 are a flexible, balloon-like material, and can comprise silicon and other suitable materials. Inflatable pads 614 can be inflated by pumping fluid to the interior of inflatable pads 614. Inflatable pads 614 can also be deflated by pumping fluid out of the interior of inflatable pads 614 or by merely relieving the fluid pressure from inside of inflatable pads 614. By inflating inflatable pads 614, compressive forces can be applied to the outer surfaces of an artery that clamp 610 surrounds. As such, clamp 610 is sized according to the size of the particular artery on which clamp 610 will be installed.

The fluid used to inflate inflatable pads 614 can be provided from a reservoir 630. Reservoir 630 can be implanted in a suitable location beneath the surface of the skin within the patient. Reservoir 630 can be flexible so that as fluid flows from reservoir 630 to inflatable pads 614, reservoir 630 can collapse in response to having a lower volume of fluid. The fluid handling system can be a closed system. Reservoir 630 can also flexibly receive fluid from inflatable pads 614 when inflatable pads 614 deflate. In some cases, the fluid used for inflation can be a saline solution, or any suitable biocompatible fluid.

The inflation and deflation of inflatable pads 614 can be accomplished by the actions of a controller 620. Controller 620 can include a reversible pump 622, a microprocessor 624, a power source 626, and an antenna 628.

Reversible pump 622 can pressurize the inflation fluid to create pressure differentials between reservoir 630 and inflatable pads 614. Reversible pump 622 can operate to cause inflation fluid to flow from reservoir 630 to inflatable pads 614, and to flow from inflatable pads 614 to reservoir 630. Reversible pump 622 can also maintain a constant pressure differential between reservoir 630 and inflatable pads 614. For example, when inflatable pars 614 are pressurized in comparison to reservoir 630, reversible pump 622 can maintain the pressure differential with no substantial pressure decay over time. In some cases, one or more fluid control valves can be included to maintain the constant pressure differential.

Microprocessor 624 can control the operations of reversible pump 622. Microprocessor 624 can receive control commands from external controllers via antenna 628. Microprocessor 624 can provide a power switching function by directing electrical power from power source 626 (e.g., lithium-iodine batteries, lithium-ion batteries, and the like) to reversible pump 622 to actuate reversible pump 622 in the desired flow direction. In some cases, the power source 626 can be one or more rechargeable batteries and the rechargeable batteries can be recharged using a wireless induction charging system.

Optionally, a safety electrode 650 can be in electrical communication with microprocessor 624. In some cases, safety electrode 650 can provide feedback to microprocessor 624 regarding the status of arterial perfusion of intestines affected by arterial band-constrictor device 600. For example, safety electrode 650 can be a pH sensor that measures the pH of the intestinal tissue. When pH of tissue drops below a threshold level, it can be an indicator that the blood flow to the tissue is insufficient to prevent ischemia, and that the amount of constriction on the artery should be reduced. In some cases, safety electrode 650 can be another suitable type of perfusion-monitoring sensor that can provide feedback to microprocessor 624 regarding the extent of perfusion of the intestinal tissues affected by arterial constrictor device 600. With the feedback provided by the optional safety electrode 650, microprocessor 624 can respond by taking various countermeasures as appropriate. In some cases, microprocessor 624 can actuate reversible pump 622 to decrease the compressive force applied by inflatable pads 614 on the artery it surrounds. In some cases, microprocessor 624 can trigger an alarm that can be received by wireless controllers, such as those described in reference to FIG. 3. In some cases, both such actions, and others, can be initiated by microprocessor 624.

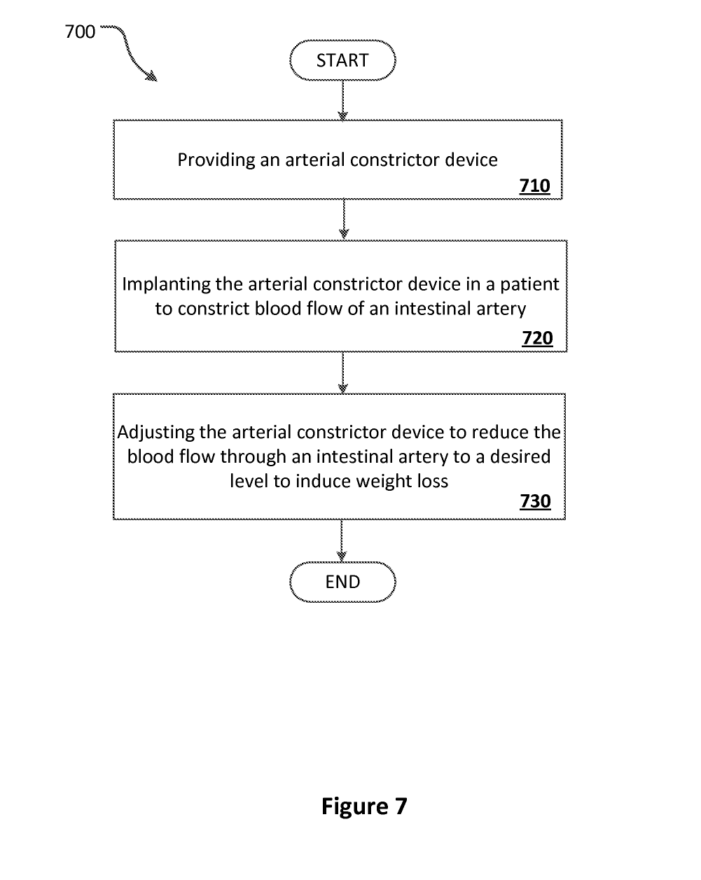

FIG. 7 illustrates a process 700 for adjustably using an arterial band-constrictor device to induce weight loss in a patient. At operation 710, an arterial constrictor device is provided. The arterial constrictor device can be configured to be placed around the outer periphery of an artery that supplies blood to digestive organs in a human patient. The arterial constrictor device can be configured to be implantable in the patient, and configured to be controllable by one or more external controllers. The external controllers can be capable of adjusting the amount of compressive force for vessel constriction that the arterial constrictor device applies to an artery supplying a digestive organ.

At operation 720, the arterial constrictor device is implanted in a patient to constrict blood flow of an intestinal artery. The arterial constrictor device can be placed around an artery (e.g., SMA) that supplies an organ of the digestive system of a patient, e.g., the small intestine. The arterial constrictor device can be configured to apply a compressive force to the outer wall of an artery. The compressive force can result in a reduction of the open area of the arterial vessel, to thereby reduce the rate of blood flow through the artery that supplies the organ of the digestive system of the patient.

At operation 730, the arterial constrictor device can be adjusted to reduce the blood flow through an intestinal artery to a desired level to induce weight loss. In some cases, the amount of constriction resulting from the arterial constrictor device can be gradually increased to reach a desired level. In some cases, the amount of constriction resulting from the arterial constrictor device can be reduced as needed in keeping with a treatment plan for a particular patient. In some cases, the amount of constriction resulting from the arterial constrictor device can be cycled from being applied for a period of time, then not applied for a period of time, and then reapplied for a period of time, and so on as desired. The adjustment process can be performed concurrently while a physician monitors the health and weight loss of the patient. In some cases, a wirelessly controlled adjustment system can provide convenience for adjusting the arterial constrictor device without invasiveness to the body of the patient.

FIGS. 8A-8C describe example intestinal arterial constrictor systems that are well-suited for installation using minimally invasive techniques. In general, the example systems can be installed percutaneously, and tunneled under the skin using image guided access. The intestinal arterial constrictor systems provided here can thereby be installed in a patient without requiring open-surgery.

FIG. 8A depicts a schematic side view of human anatomy 800 in the abdominal region. In general, the anatomy 800 includes vertebrae of a spine 802 and an abdominal aorta 804. Branches from abdominal aorta 804 include a celiac artery 806, an SMA 808, and an inferior mesenteric artery 810.

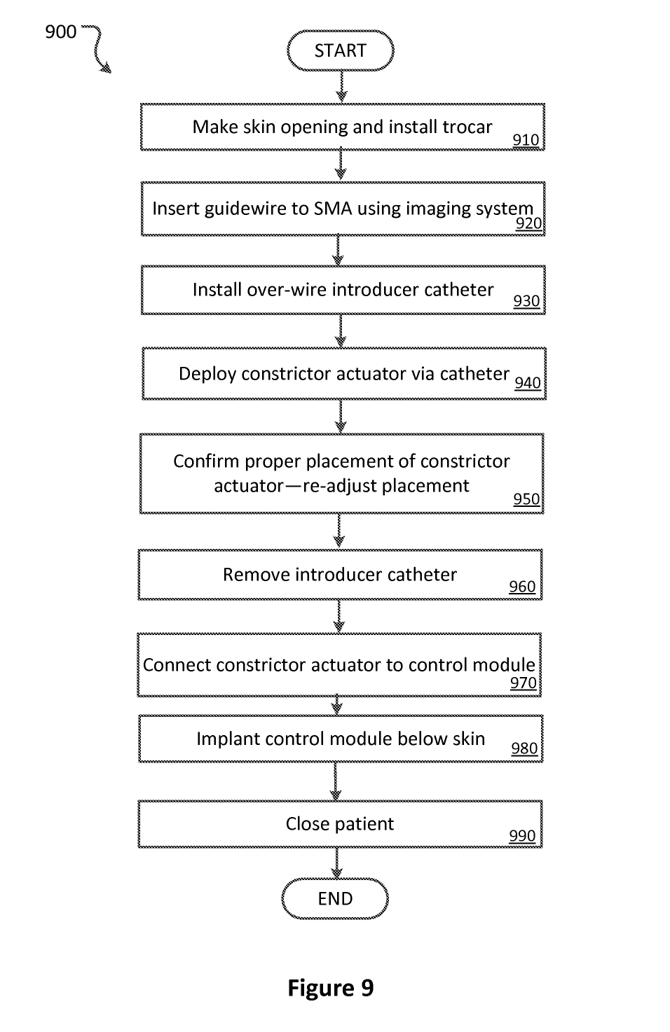

The distal portion of an arterial constrictor system 812 is schematically represented in FIG. 8A (e.g., representing systems 820 and 850 of FIGS. 8B and 8C). The distal portion of arterial constrictor system 812 is wrapped around SMA 808. This type of installation can be performed using a catheter-based deployment system under image guidance as described with reference to FIG. 9. These techniques can be used to install arterial constrictor system 812 at any suitable location on SMA 808 (e.g., as described herein in reference to FIG. 2).

FIG. 8B provides an example arterial constrictor system 820 for installation on an artery such as SMA 808. In general, constrictor system 820 includes a reinforcing member 810, an inflatable member 814, a controller 820, and a reservoir 830, that are interconnected by a flexible tubing 840. In some cases, arterial constrictor system 820 is fully implantable in a patient's body. As such, all materials are biocompatible.

Reinforcing member 810 can be configured with a C-shape to thereby partially surround an artery. Inflatable member 814 is attached to the inner-curvature of reinforcing member 810. In some cases, reinforcing member 810 and inflatable member 814 are both generally C-shaped. The C-shaped assembly of reinforcing member 810 and inflatable member 814 can be positioned to partially surround an artery, and to apply a radial force on the outer surface of the artery. The radial force applied on the outer surface of the artery can cause a constriction of the blood flow through the artery. In the case of an intestinal artery, the constriction of blood flow through the artery can result in a reduction of digestive efficiency and food adsorption, and consequently cause weight loss.

Reinforcing member 810 can be made from a flexible material so that it can be elastically deformed into a generally straight configuration, as required for being deployed through a catheter. In some cases, reinforcing member 810 is made from nitinol. Nitinol is a super-elastic material that can enable reinforcing member 810 to be elastically deformed into a straight configuration for insertion in a delivery catheter. Then, upon emergence from the delivery catheter, reinforcing member 810 will resume its natural C-shape (in a position around an artery, e.g., SMA 808). In some cases, a nitinol reinforcing member 810 can be heat-set into the C-shape as desired. In some cases, a shape-memory material (e.g., nitinol, etc.) can be used to make reinforcing member 810. In such cases, body heat can activate the shape-memory properties of reinforcing member 810 such that it assumes a C-shape in situ. In some cases, other materials are used to make reinforcing member 810, including but not limited to, stainless steel, titanium, titanium alloys, and polymeric materials.

Other features can be included to enhance the ease-of-installation and performance of the arterial constrictor system 820. For example, radio-opaque markers can be included on reinforcing member 810 and/or inflatable member 814 to assist with radiographic visualization during installation. Anchor devices can be included on reinforcing member 810 (e.g., barbs, hooks, protrusions, etc.).

In some cases, no reinforcing member 810 is included. That is, inflatable member 814 can be C-shaped and deployed around an artery without a need for reinforcing member 810.

In some cases, inflatable member 814 surrounds about 180 degrees of an artery (e.g., SMA 808). In some cases, inflatable member 814 surrounds about 180 degrees to about 210 degrees of an artery. In some cases, inflatable member 814 surrounds about 200 degrees to about 230 degrees of an artery. In some cases, inflatable member 814 surrounds about 220 degrees to about 250 degrees of an artery.

In some cases, inflatable member 814 surrounds about 240 degrees to about 270 degrees of an artery. In some cases, inflatable member 814 surrounds more than 270 degrees of an artery.

The other components of example arterial constrictor system 820 are generally analogous to similar embodiments provided herein (e.g., arterial band-constrictor system 400 of FIG. 4 and arterial constrictor system 600 of FIG. 6). The principles of operation are also generally analogous. However, arterial constrictor system 820 is well-suited to being installed percutaneously because reinforcing member 810 and inflatable member 814 can be configured in a low-profile, generally linear, configuration for insertion in a delivery catheter.

FIG. 8C provides an example arterial constrictor system 850 for installation on an artery such as SMA 808 (as well as other suitable arteries and arterial branches). In general, constrictor system 850 includes one or more electrical leads 860 that is electrically connected to a power controller 870. In some cases, arterial constrictor system 850 is fully implantable in a patient's body. As such, all materials are biocompatible.

In some cases, electrical lead 860 is wrapped onto the periphery of an intestinal artery (e.g., SMA 808). In some cases, electrical lead 860 can be wrapped fully around SMA 808. In some cases, electrical lead 860 can be partially wrapped around SMA 808. In some cases, electrical lead 860 merely makes contact with a portion of the outer surface of SMA 808. In some cases, two or more electrical leads 860 are installed on SMA 808, or on various other abdominal arteries.

Electrical lead 860 can be supplied with electrical current and controlled by power controller 870 that can include a battery-pack energy source. When electromotive stimulation pulses are transferred from power controller 870 via electrical lead 860 to SMA 808, a restriction of blood flow can result from contraction of SMA 808. Power controller 870 can be programmed to modulate and intermittently apply electromotive stimulation pulses in a suitable pattern. In some cases, power controller 870 can be wirelessly controlled and programmed using the systems and techniques described herein, for example in reference to FIG. 3.

FIG. 9 is a flowchart of a method 900 for percutaneously installing abdominal arterial constrictor systems (such as the systems 820 and 850 of FIGS. 8B and 8C). In general, method 900 uses image guided access and an over-the-wire catheter-based approach.

At operation 910, an opening such as an incision is made to the skin of the patient, and a trocar is optionally installed. In some cases, it may be effective to enter the patient's abdominal area using a transperitoneal or retroperitoneal approach. In some cases, a front or rear abdominal entry can be used.

At operation 920, a guidewire can be installed through the skin opening/trocar such that the distal end of the guidewire is near the target site where the constrictor device will be installed, and the proximal end of the guidewire is external of the patient. The guidewire can be tunneled under the skin. In some cases, a tunnel under the patient's skin can be created by performing blunt dissection prior to insertion of the guidewire. The guidewire can be installed using image guided access. For example, ultrasound, fluoroscopy, or computed tomography (CT) can be used to provide visualization of the guidewire placement. Other imaging system technologies can also be used. In some cases, a catheter is installed without the prior installation of a guidewire.

At operation 930, an introducer catheter is installed over the guidewire. The distal tip of the catheter is positioned neat the target site at the artery, and the proximal end is external of the patient. In some cases, the guidewire can then be removed. In some cases, the guidewire can be left in the catheter and the guidewire can be used for various other purposes. In some cases, the catheter or a portion of the catheter is steerable.

At operation 940, the constrictor device is inserted in the catheter and pushed through the catheter to the target site at the artery. In some cases, a pusher-catheter is used to push the constrictor device through the catheter. In some cases, the constrictor device has enough column strength to be pushed without additional implements. As the constrictor device emerges from the distal tip of the catheter, it can be maneuvered to make proper contact with the target artery as desired. For example, the constrictor device may be partially or fully wrapped around the periphery of the artery. The constrictor device may be configured with a natural tendency to wrap partially or fully around the artery. Radio-opaque markers may be included on the constrictor device to make the orientation of the constrictor device more radiographically visible.

At operation 950, the clinician operator can confirm proper placement of the constrictor device and make adjustments as needed. The imaging system (CT, ultrasound, fluoroscopy, etc.) can be used for assisting in this step. In some cases, the constrictor device can be retracted into the introducer catheter, and then redeployed to reposition the constrictor device on the artery as desired. In some cases, the introducer catheter can be steerable or maneuverable to assist with such placement of the constrictor device.

After confirmation of proper placement of the constrictor device on the target artery, at operation 960 the introducer catheter (and trocar) can be removed from the patient. At operation 970, a control module can be connected to the constrictor device. At operation 980, a pocket can be created under the skin of the patient, and the control module can be installed in the pocket under the skin of the patient. At operation 990, the openings of the patient's skin can be closed.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A method for reducing caloric uptake of a mammal, wherein said method comprises:
(a) implanting an arterial constrictor device in said mammal, wherein said arterial constrictor device is configured to adjustably apply a compressive force on an outer surface of an artery that supplies blood to an intestinal organ; and
(b) adjusting said compressive force to modulate blood flow to said intestinal organ to a desired level.

2. The method of claim 1, wherein said mammal is a human.

3. The method of claim 2, wherein said artery that supplies blood to an intestinal organ is a superior mesenteric artery, celiac axis artery, inferior mesenteric artery, or branches of said arteries.

4. The method of claim 1, wherein said adjusting said compressive force to modulate said blood flow to said intestinal organ includes periods of time when said compressive force is adjusted to be substantially zero.

5. A system for adjustably constricting an arterial vessel that supplies blood to an intestinal organ in a mammal, said system comprising:
(a) a constrictor device configured for implantation in said mammal, said constrictor device configured to substantially surround an outer periphery of said arterial vessel and to adjustably apply a compressive force on said outer periphery, wherein said constrictor device is configured to wirelessly receive control commands that are capable of causing said compressive force to be increased and to wirelessly receive control commands that are capable of causing said compressive force to be decreased, and wherein said constrictor device is configured to adjust said compressive force in response to said control commands; and
(b) an external controller configured to wirelessly send said control commands for causing said compressive force to be adjusted.

6. The system of claim 5, wherein said mammal is a human.

7. The system of claim 5, comprising a sensor configured to assess a level of perfusion of said intestinal organ and to provide to said constrictor device a signal corresponding to said level of perfusion.

8. The system of claim 7, wherein said sensor is a pH sensor.

9. The system of claim 7, wherein said constrictor device is configured to adjust said compressive force in response to said signal.

10. A system for adjustably constricting an arterial vessel that supplies blood to an intestinal organ in a mammal, said system comprising:
(a) a constrictor device configured for implantation in said mammal using a percutaneous catheter-based technique, said constrictor device configured to at least partially surround an outer periphery of said arterial vessel and to adjustably apply a compressive force on at least a portion of said outer periphery, wherein said constrictor device is configured to wirelessly receive control commands that are capable of causing said compressive force to be increased and to wirelessly receive control commands that are capable of causing said compressive force to be decreased, and wherein said constrictor device is configured to adjust said compressive force in response to said control commands; and
(b) an external controller configured to wirelessly send said control commands for causing said compressive force to be adjusted.

11. The system of claim 10, wherein said mammal is a human.

12. The system of claim 10, comprising a sensor configured to assess a level of perfusion of said intestinal organ and to provide to said constrictor device a signal corresponding to said level of perfusion.

13. The system of claim 10, wherein said sensor is a pH sensor.

14. The system of claim 10, wherein said constrictor device is configured to adjust said compressive force in response to said signal.

15. A method for percutaneously installing a system for adjustably constricting an arterial vessel that supplies blood to an intestinal organ in a mammal, said method comprising:
(a) inserting a guidewire through an opening in said mammal's skin and using an imaging system to maneuver a distal tip of said guidewire to a target location on said arterial vessel;
(b) installing a catheter over said guidewire;
(c) inserting at least a portion of said system into said catheter;
(d) causing a distal end of said system to emerge from said catheter at said target location, wherein at least a portion of said distal end of said system wraps around at least a portion of a periphery of said arterial vessel, wherein said system is configured to partially surround said at least a portion of a periphery of said arterial vessel and to adjustably apply a compressive force on said at least a portion of a periphery, wherein said constrictor device is configured to wirelessly receive control commands that are capable of causing said compressive force to be increased and to wirelessly receive control commands that are capable of causing said compressive force to be decreased, and wherein said constrictor device is configured to adjust said compressive force in response to said control commands; and
(e) implanting a control module of said system under said mammal's skin.

16. The method of claim 15, wherein said mammal is a human.

17-21. (canceled)
